(19) United States
(12) Patent Application Publication
(10) Pub. No.: US 2025/0344964 A1
(43) Pub. Date: Nov. 13, 2025

(54) PULMONARY DISEASE SCREENING SYSTEM AND METHOD FOR SCREENING PULMONARY DISEASES USING SAME

(71) Applicant: Electronics and Telecommunications Research Institute, Daejeon (KR)

(72) Inventors: Joon-Hak BANG, Daejeon (KR); SUNG JUNE CHANG, Daejeon (KR); Jong-woo CHOI, Daejeon (KR)

(73) Assignee: Electronics and Telecommunications Research Institute, Daejeon (KR)

(21) Appl. No.: 18/947,117

(22) Filed: Nov. 14, 2024

(30) Foreign Application Priority Data
May 9, 2024 (KR) 10-2024-0061124

(51) Int. Cl. A61B 5/097; A61B 5/08; G01N 33/497

(52) U.S. Cl. CPC A61B 5/097; A61B 5/082; G01N 33/4975

(57) ABSTRACT

Proposed is a pulmonary disease screening system and a method for screening pulmonary disease using the same, which is configured to integrate a lung function measurement device and a breath analysis device into a single device and analyze biomarkers in pulmonary diseases by extracting the alveolar gas, the last part of the expired air injected into a lung function measurement device.

### CROSS REFERENCE TO RELATED APPLICATION

The present application claims priority to Korean Patent Application No. 10-2024-0061124,filed on May 9, 2024, the entire contents of which are incorporated herein for all purposes by this reference.

### BACKGROUND

### Technical Field

The present disclosure relates to a pulmonary disease screening system and a method for screening pulmonary disease using the same, which improves the accuracy of pulmonary disease screening by extracting the alveolar gas from a person's exhalation.

### Description of the Related Art

Non-invasive pulmonary disease screening refers to the process of detecting and taking measures on lung-related diseases at an early stage, and pulmonary disease screening methods can be broadly categorized into methods using chest X-rays, computed tomography, exhaled breath tests, and blood oxygen levels measurements.

Of these, pulmonary disease screening, which diagnoses pulmonary diseases by analyzing the expired air a person exhales, is one of the non-invasive methods of evaluating health conditions by analyzing the gas contents of the expired air, and refers to identifying biomarkers of a specific disease by measuring the components in the patient's exhaled breath.

That is, a human's exhaled breath may contain numerous biometric information about health conditions and diseases, and pulmonary disease screening can roughly diagnose not only pulmonary disease but also periodontal disease, fat burning, diabetes, kidney disease, colon disease, Alzheimer's, and the like by using a biomarker analysis technology that contains various volatile organic compounds (VOCs) emitted from exhaled breath.

Exhaled gas refers to a gas discharged through the oral or nasal cavity in the exhaled breath of the respiratory action of the human body and contains various gas components related to the metabolism of the human body.

At this time, a breath analysis device as a device that analyzes these exhaled gases can measure the components and concentration of a specific gas in the exhaled gas and then with the measured values can roughly estimate diseases of the human body.

However, conventional breath analysis devices have developed to the extent of analyzing the gas generated in the oral cavity related to bad breath and the gas composition generated in the bronchial tubes related to asthma, so it is not possible to analyze the alveolar gas composition generated within the deep lungs related to lung disease.

Therefore, improving the accuracy of pulmonary disease diagnosis with pulmonary disease screening using the conventional breath analysis device is difficult.

In addition, pulmonary disease screening using the conventional breath analysis device requires measuring lung functions such as expiratory volume, vital capacity, and lung volume for further pulmonary disease screening separately from breath analysis, thereby resulting in a problem that a separate lung function measurement device 20 is additionally required as shown in FIG. 1 in addition to the breath analysis device 10 described above.

### DOCUMENTS OF RELATED ART

(Patent Document 1) Korean Patent Application Publication No. 10-2023-0136818

### SUMMARY

An objective of the present disclosure is to provide a pulmonary disease screening system and a method for screening pulmonary disease using the same, which is capable of analyzing biomarkers in pulmonary diseases by extracting the alveolar gas, the last part of the expired air a person exhales.

Another objective of the present disclosure is to provide a pulmonary disease screening system and a method for screening pulmonary disease using the same, which is capable of performing both breath analysis and lung function measurement by integrating breath analysis and lung function measurement into a single device.

Another objective of the present disclosure is to provide a pulmonary disease screening system and a method for screening pulmonary disease using the same, which includes an appropriate control of a valve capable of cutting off the last part of the expired air for the extraction and analysis of the alveolar gas to be analyzed, a sampling loop capable of accommodating the same, and a pump control for constantly flowing the alveolar gas to a sensor.

In order to achieve the objectives of the present disclosure, the present disclosure includes a lung function measurement line forming a flow path for an expired air a patient exhales to pass through and including a first flow sensor for measuring a flow rate of the expired air, a first branch flow path branched from the lung function measurement line, an alveolar gas extraction line, which includes a first valve connected to the first branch flow path and a first pump for generating a suction force, and forms a flow path for extracting and passing a part of the expired air passing through the lung function measurement line with the suction force of the first pump, a second branch flow path branched from the alveolar gas extraction line, and an analysis line, which includes a third valve connected to the second branch flow path and a second pump for generating a suction force and analyzes a biomarker in pulmonary diseases by passing an alveolar gas extracted from the alveolar gas extraction line through the second branch flow path with the suction force of the second pump, wherein the first valve and the third valve are controlled to switch a flow of the expired air passing through the alveolar gas extraction line to the analysis line when the flow rate of the expired air measured through the first flow sensor is “0”.

At this time, a second valve for switching the flow of the expired air of the alveolar gas extraction line to the analysis line should be further formed between the first valve and the second branch flow path.

At this time, a sampling loop for collecting and accommodating the expired air passing through the alveolar gas extraction line should be further formed between the first valve and the second valve.

In this case, the alveolar gas extraction line should further include a second flow sensor for measuring the flow rate of the expired air introduced into the sampling loop.

In addition, the analysis line should further form an outside air inflow path capable of introducing outside air through the third valve.

At this time, a bypass flow path for diverting an inflow direction of outside air to the alveolar gas extraction line should be further formed between the outside air inflow path and the first valve.

In addition, the analysis line should further include a measurement unit for measuring the biomarker in pulmonary diseases.

At this time, the measurement unit should include an NO sensor for measuring nitric oxide, which is the biomarker in pulmonary diseases, a temperature and humidity sensor for measuring temperature and humidity around the NO sensor, and a third flow sensor for monitoring whether the alveolar gas flows to the NO sensor at a constant flow rate.

In addition, a Nafion tube for reducing a humidity of the alveolar gas flowing into the measurement unit should be further formed between the third valve and the measurement unit.

In addition, the first valve and the second valve should be controlled to switch the flow of the expired air of the alveolar gas extraction line only toward the analysis line, when the flow rate of the expired air measured through the first flow sensor is “0”.

In addition, when an amount of the expired air measured through the first flow sensor is measured as “0” in a state where the third valve is controlled to close the second branch flow path and open the outside air inflow path, the first valve may be controlled to open the bypass flow path, the second valve may be controlled to open the second branch flow path, and the third valve should be controlled to close the outside air inflow path and open the second branch flow path.

In addition, the first valve, the second valve, and the third valve should be three-way valves.

As another example to achieve the objectives, disclosed is a pulmonary disease screening system which includes a lung function measurement line forming a flow path for an expired air a patient exhales to pass through and including a first flow sensor for measuring a flow rate of the expired air, a first branch flow path branched from the first flow sensor, an alveolar gas extraction line including a first valve connected to the first branch flow path, a first pump for generating a suction force, a second valve formed between the first pump and the first valve, a sampling loop formed between the first valve and the second valve to capture an alveolar gas, a second flow sensor formed between the first pump and the second valve to measure the flow rate, a second branch flow path branched from the second valve, and an analysis line including a third valve connected to the second branch flow path, a second pump for generating a suction force, and a measurement unit formed between the third valve and the second pump to measure a biomarker in pulmonary diseases of the expired air passing through the third valve, wherein the first valve and the second valve are controlled to extract the expired air passing through the lung function measurement line to the alveolar gas extraction line and then the first, second, and third valves are controlled to switch a flow of the expired air of the alveolar gas extraction line to the analysis line when the flow rate of the expired air measured by the first flow sensor is “0”.

At this time, the analysis line should further include an outside air inflow path capable of introducing outside air through the third valve, and a bypass flow path formed between the outside air inflow path and the first valve to divert an inflow direction of outside air to the alveolar gas extraction line.

At this time, when the flow rate of the expired air is measured as “0” through the first flow sensor in a state where the first valve and the second valve are controlled to open only the first branch flow path and the flow path of the alveolar gas extraction line and the third valve is controlled to open only the outside air inflow path and the analysis line, the first valve is controlled to close the first branch flow path and open the bypass flow path, the second valve is controlled to allow the expired air to be discharged only toward the second branch flow path, and the third valve is controlled to close the outside air inflow path and open the second branch flow path.

As another example to achieve the objectives, a pulmonary disease screening method for extracting an expired air a patient exhales and analyzing the expired air is disclosed, wherein the method performs pulmonary disease screening by extracting an alveolar gas, which is the last part of the expired air, when a flow rate of the expired air becomes “0” and by analyzing a biomarker in pulmonary diseases.

At this time, an alveolar gas extraction from the expired air should be performed through a flow detection of a flow sensor, an expired air capture of a sampling loop, a power of a pump, and a switching control of a valve.

At this time, the method includes (a) injecting the expired air the patient exhales into a lung function measurement line and continuously introducing the outside air into an analysis line, (b) measuring the flow rate of the expired air the patient exhales into the lung function measurement line by a manager, (c) extracting the expired air passing through the lung function measurement line into an alveolar gas extraction line, (d) discharging the alveolar gas extracted from the alveolar gas extraction line to the analysis line by switching the flow of the expired air of the alveolar gas extraction line to the analysis line and by diverting a flow of the outside air to the alveolar gas extraction line by the analysis line when the flow rate of the expired air becomes “0” in the step (b), and (e) analyzing the biomarkers in pulmonary diseases in the alveolar gas introduced from the alveolar gas extraction line by the analysis line.

The effects of the present disclosure obtained through the solution means described above are as follows.

First, the present disclosure has the effect of improving the accuracy of pulmonary disease analysis by extracting and analyzing the alveolar gas, the last part of the expired air, in screening pulmonary diseases by using the expired air, which is a non-invasive method.

Second, the present disclosure has the effect of reducing the cumbersome of the measurement and analysis process and reducing the burden of additional costs, by integrating lung function measurement and breath analysis into one system.

Third, the present disclosure has the effect of stabilizing and facilitating the patient's respiration for extracting the alveolar gas, by extracting into an alveolar gas extraction line only the alveolar gas, the last part of the expired air, using the pump power of the alveolar gas extraction line, when a patient naturally exhales into the lung function measurement line rather than the patient directly exhales into the alveolar gas extraction line in the process of extracting the alveolar gas from the expired air.

### BRIEF DESCRIPTION OF THE DRAWINGS

FIG. 1 is a view schematically showing a state where each of a breath analysis using a breath analysis device according to a conventional technology and a lung function measurement using a lung function measurement device is respectively performed.

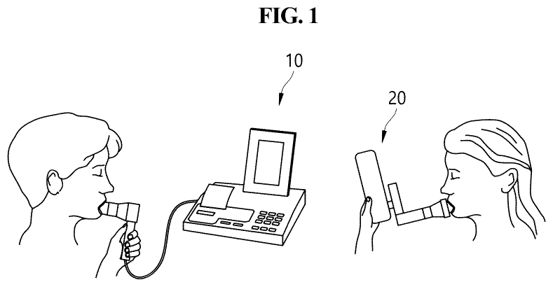

FIG. 2 is a block diagram showing a pulmonary disease screening system according to a preferred exemplary embodiment of the present disclosure.

FIG. 3 is a block diagram showing a pulmonary disease screening system according to a preferred exemplary embodiment of the present disclosure, which shows a flow of exhaled gas during an operation of measuring lung function.

FIG. 4 is a block diagram showing a pulmonary disease screening system according to a preferred exemplary embodiment of the present disclosure, which shows a flow of exhaled gas during an operation of extracting alveolar gas.

FIG. 5 is a block diagram showing a pulmonary disease screening system according to a preferred exemplary embodiment of the present disclosure, which shows a flow of exhaled gas during an operation of analyzing alveolar gas.

FIG. 6 is a view schematically showing the switching states of a first, second, and third valves when performing an alveolar gas extraction operation of a pulmonary disease screening system according to a preferred exemplary embodiment of the present disclosure.

FIG. 7 is a view schematically showing the switching states of a first, second, and third valves when performing an alveolar gas analysis operation of a pulmonary disease screening system according to a preferred exemplary embodiment of the present disclosure.

FIG. 8 is a flowchart of a pulmonary disease screening method according to a preferred exemplary embodiment of the present disclosure.

### DETAILED DESCRIPTION OF THE DISCLOSURE

Hereinafter, a pulmonary disease screening system and a method for screening pulmonary diseases using the same will be described in more detail with reference to the drawings.

In describing exemplary embodiments disclosed in the present specification, the detailed

description will be omitted when it is determined that a detailed description of related known technology may obscure the gist of the exemplary embodiments disclosed in the present specification.

The attached drawings are only intended to facilitate understanding of the exemplary embodiments disclosed in this specification, and the technical ideas disclosed in this specification are not limited by the attached drawings, and should be understood to include all modifications, equivalents, or substitutes included in the spirit and technical scope of the present disclosure.

In the following description, a singular expression includes a plural expression unless the context clearly indicates otherwise.

In this application, it should be understood that terms such as “include” or “have” are intended to specify the presence of a feature, number, step, operation, component, part or combination thereof described in the specification, but do not exclude in advance the possibility of the presence or addition of one or more other features, numbers, steps, operations, components, parts or combinations thereof.

As used herein, including in the claims, “or” as used in a list of items (e.g., a list of items prefaced by a phrase such as “at least one of” or “one or more of” or “one or both of”) indicates an inclusive list such that, for example, a list of at least one of A, B, or C means A or B or C or AB or AC or BC or ABC (i.e., A and B and C).

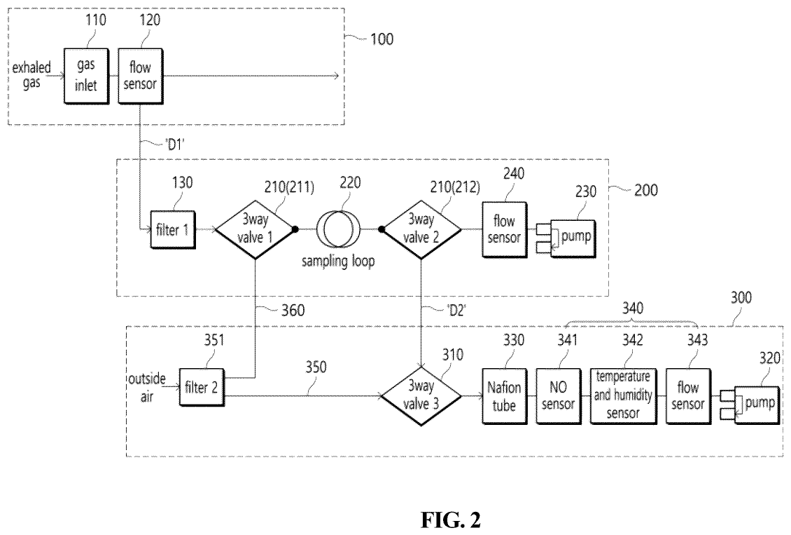

Hereinafter, a pulmonary disease screening system according to a preferred exemplary embodiment of the present disclosure will be described with reference to FIG. 2.

The pulmonary disease screening system may integrate a device for analyzing expired air and a device for measuring lung function into a single system, which extracts and analyzes the alveolar gas, the last part of expired air, among exhaled gas.

Accordingly, the pulmonary disease screening system may relieve the cumbersome caused by pulmonary disease screening and may improve the accuracy of pulmonary disease analysis.

As shown in FIG. 2, the pulmonary disease screening system may include a lung function measurement line 100, an alveolar gas extraction line 200, and an analysis line 300.

The lung function measurement line 100 may form a flow path for the exhaled gas (exhaled breath) a patient exhales to pass through, wherein one side of the flow path forms a gas inlet 110, and the other side of the flow path forms a gas outlet. In this case, the gas inlet 110 may be a part where the patient puts his/her mouth, and a mouthpiece (not shown) may be formed at the gas inlet 110 for the convenience of the patient.

That is, the flow path of the lung function measurement line 100 may form a flow path through which the patient's exhaled gas is introduced and then discharged to the outside.

In this case, the lung function measurement line 100 may further form a flow sensor on the flow path for passing the exhaled gas. The flow sensor may be referred to as a first flow sensor 120 for convenience of description.

The first flow sensor 120 may measure the flow rate of the exhaled gas passing through the lung function measurement line 100, and a known technique may be applied to the measurement structure and method.

In this case, a filter 130 for filtering foreign substances contained in the exhaled gas may be further formed between the first flow sensor 120 and the gas inlet 110. That is, as a sampling gas for pulmonary disease analysis, the expired air measured through the first flow sensor 120 should be pure exhaled gas without foreign substances, such that the filter 130 can filter out saliva or foreign substances contained in the exhaled gas of the patient and allow only pure exhaled gas to pass.

In addition to measuring the patient's lung function, the first flow sensor 120 may measure the flow rate of the patient's expired air in order to extract the alveolar gas, the last part of the exhaled gas, that is, which allows a manager to recognize that the last part of the expired air, the alveolar gas, is being exhaled when the value of the flow rate of the expired air measured through the first flow sensor 120 becomes “0”.

Next, the alveolar gas extraction line 200 may serve to switch the flow of the expired air to the analysis line 300 by extracting the expired air of the patient passing through the lung function measurement line 100.

In particular, the alveolar gas extraction line 200 may discharge to the analysis line 300 the alveolar gas, the last part of the expired air, passing through the lung function measurement line 100, thereby improving the accuracy of pulmonary disease screening and forming a flow path for passing the expired air.

The alveolar gas extraction line 200 may include a valve 210, a sampling loop 220, a pump 230, and a flow sensor 240.

The valve 210 may serve to open and close each of the flow path of the alveolar gas extraction line 200, the lung function measurement line 100, and the analysis line 300 to be described later.

The valve 210 may include a first valve 211 and a second valve 212.

The first valve 211 may serve to allow the flow path of the lung function measurement line 100 to communicate with the flow path of the alveolar gas extraction line 200, and may serve to allow the flow path of the alveolar gas extraction line 200 to communicate with the outside air inflow path of the analysis line 300 to be described later.

In addition, the first valve 211, in conjunction with the second valve 212, may serve to switch the flow of the expired air passing through the lung function measurement line 100 to the alveolar gas extraction line 200.

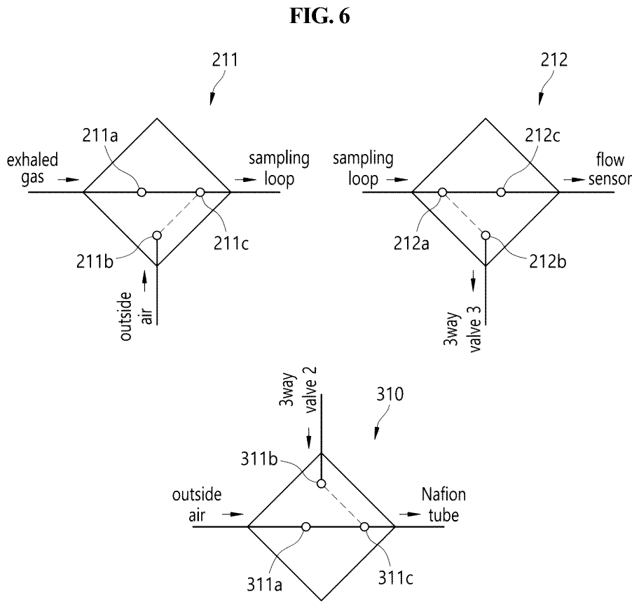

The first valve 211 for performing this role may be formed as a three-way solenoid valve. In this case, the first valve 211 may include a first inlet 211a, a second inlet 211b, and an outlet 211c, as shown in FIG. 6.

The second valve 212 may serve, in conjunction with the first valve 211, to switch the flow of the expired air passing through the lung function measurement line 100 to the alveolar gas extraction line 200 and to switch the alveolar gas, the last part of the expired air, to the analysis line, and may be formed on the flow path of the alveolar gas extraction line 200.

The second valve 212 may also be formed as a three-way solenoid valve. In this case, the second valve 212 may include an inlet 212a, a first outlet 212b, and a second outlet 212c, as shown in FIG. 6.

The sampling loop 220 may serve to trap and temporarily store the alveolar gas extracted from the lung function measurement line 100, and may be formed between the first valve 211 and the second valve 212.

The sampling loop 220 may be formed to introduce the exhaled gas switched from the lung function measurement line 100 through the first valve 211 and then discharge the same through the second valve 212.

As described above, the present disclosure may be to analyze pulmonary diseases by extracting the patient's alveolar gas, and the sampling loop 220 may be configured to capture the alveolar gas, the last part of the expired air, for pulmonary disease analysis.

The sampling loop 220 may have an effect of storing only the alveolar gas, the last part of the expired air, by artificially forming the flow path, through which the exhaled gas passes, to be thin and long and by preventing the front part of the expired air from mixing with the back part in the flow of time.

To this end, the sampling loop 220 may be formed in a plurality.

The pump 230 may generate the power for extracting the expired air from the lung function measurement line 100 by generating the suction force in the flow path of the alveolar gas extraction line 200.

That is, the present disclosure may enable the patient's exhaled gas to be extracted by using the power of the pump 230 and since the orifices (not shown) formed in the first valve 211 and the second valve 212 have a small diameter, it may not be possible to introduce a flow rate of an appropriate standard for measuring lung function and measuring the alveolar gas when the patient directly exhales the expired air toward the first valve 211, so the present disclosure may enable the exhaled gas to be easily and stably extracted by using the power of the pump 230. A detailed description thereof will be described later.

The pump may be referred to as a first pump 230 for convenience of description. The first pump 230 may operate when the pulmonary disease screening mode starts.

The flow sensor 240 may serve to measure the flow rate of the expired air flowing through the alveolar gas extraction line 200, and may be referred to as a second flow sensor 240 for convenience of description.

The second flow sensor 240 may be configured to extract a desired amount of the patient's expired air passing through the lung function measurement line 100, and may provide the expired air without shortage for the screening analysis to the analysis line 300 by measuring the flow rate of the expired air passing through the sampling loop 220.

The second flow sensor 240 may be provided between the first pump 230 and the second valve 212, and a known technique may be applied to the flow rate measurement and method.

Meanwhile, a flow path for communicating the flow of the alveolar gas may be formed between the lung function measurement line 100 and the alveolar gas extraction line 200, and the flow path may be referred to as a first branch flow path (D1) for convenience of description.

The first branch flow path D1 may be formed between the first flow sensor 120 of the lung function measurement line 100 and the first valve 211 of the alveolar gas extraction line 200.

Next, the analysis line 300 may serve to analyze the biomarkers in pulmonary diseases of the alveolar gas contained in the last part of the expired air extracted through the alveolar gas extraction line 200.

For example, the biomarkers related to pulmonary diseases such as chronic obstructive pulmonic disease (COPD), pulmonary fibrosis, pneumonia and the like in the exhaled gas may be known as nitric oxide (NO), and the analysis line 300 may analyze pulmonary diseases by measuring the concentration of nitric oxide contained in the alveolar gas, the last part of the expired air extracted through the alveolar gas extraction line 200.

The analysis line 300 may include a valve 310, a pump 320, a Nafion tube 330, and a measurement unit 340.

The valve 310 may serve to open and close the second branch flow path D2 to be described later in order to introduce the last part of the expired air extracted through the alveolar gas extraction line 200 into the analysis line 300, or serve to open and close the outside air inflow path 350 to be described later in order to introduce outside air, and may be referred to as a third valve 310 for convenience of description.

In this case, the third valve 310 may also be formed as a three-way solenoid valve. In this case, the third valve 310 may include a first inlet 311a, a second inlet 311b, and an outlet 311c, as shown in FIG. 6.

The pump 320 may generate a suction force in the flow path of the analysis line 300 to generate the flow of gas at a constant flow rate in the analysis line 300.

The pump may be referred to as a second pump 320 for convenience of description.

The Nation tube 330 may form a flow path through which the alveolar gas introduced into the analysis line 300 through the third valve 310 passes, and may serve to minimize the moisture contained in the alveolar gas before the alveolar gas is introduced into the measurement unit 340.

The material of the Nafion tube 330 may be made of Nafion, and since it is made of a material that absorbs moisture, the measurement accuracy and stability of the measurement unit 340 may be improved by reducing the moisture of the alveolar gas passing through the measurement unit 340.

The Nation tube 330 may be formed between the third valve 310 and the measurement unit 340.

The measurement unit 340 may measure the alveolar gas introduced into the analysis line 300, and may include an NO sensor 341, a temperature and humidity sensor 342, and a flow sensor 343.

The NO sensor 341 may detect and measure nitric oxide, which is a biomarker related to pulmonary diseases, and may determine that there is an abnormality related to pulmonary diseases such as COPD, lung fibrosis, and pneumonia when the concentration of nitric oxide measured through the NO sensor 341 exceeds the reference value.

The temperature and humidity sensor 342 may serve to monitor the temperature and humidity of the alveolar gas.

Because of the characteristics of the NO sensor 341, the measurement value may vary depending on changes in the temperature and humidity of the alveolar gas, so the system may monitor the temperature and humidity of the alveolar gas through the temperature and humidity sensor 342 since the measurement value of the NO sensor 341 needs to be corrected depending on the changes in the temperature and humidity of the alveolar gas.

The flow sensor 343 may serve to measure the flow rate of the alveolar gas flowing into the NO sensor 341 in order to confirm whether the flow rate of the alveolar gas flowing into the NO sensor 341 is constantly maintained at an appropriate intensity. For convenience of description, the flow sensor may be referred to as a third flow sensor 343.

Because of the characteristics of the NO sensor 341, the measurement value may vary depending on the changes in the flow rate of the flowing alveolar gas, so the system may monitor the flow rate of the alveolar gas through the flow sensor 343 since it is necessary to confirm whether the alveolar gas flows at a constant flow rate and to adjust the strength of the second pump 320 to a preset flow rate when the flow rate varies.

Meanwhile, a branch flow path may be formed between the analysis line 300 and the alveolar gas extraction line 200, which is a flow path that introduces the last part of the expired air extracted from the alveolar gas extraction line 200 into the analysis line 300. For convenience of description, the branch flow path may be referred to as a second branch flow path D2.

The second branch flow path D2 may be formed between the second valve 212 and the third valve 310.

In addition, the analysis line 300 may form an outside air inflow path 350 for introducing outside air.

The outside air inflow path 350 may form a flow path capable of introducing outside air into the flow path of the analysis line 300, and the outside air may be introduced into the outside air inflow path 350 through the power of the second pump 320.

The analysis line 300 may continuously introduce outside air through the outside air inflow path 350 and always maintain a constant intensity of fluid flow in the analysis line 300.

At this time, the NO sensor 341 may operate unstable when detecting a sudden change in the flow rate, so by ensuring that a constant flow of fluid is always maintained in the analysis line 300 through the outside air inflow path 350, it may be possible to minimize the effect of the change in the flow rate affecting on the NO sensor 341 even when a sudden change in the flow rate occurs on account of the alveolar gas flowing into the analysis line 300 from the alveolar gas extraction line 200.

In addition, the outside air inflow path 350 may be formed in the third valve 310, and the outside air inflow path 350 may further form a filter 351 that filters out external dust or foreign substances.

Meanwhile, a bypass flow path 360 may be further formed between the outside air inflow path 350 and the alveolar gas extraction line 200.

The bypass flow path 360 may be configured to divert the outside air introduced through the outside air inflow path 350 to the alveolar gas extraction line 200 and transfer the alveolar gas extracted from the alveolar gas extraction line 200 to the analysis line 300.

The bypass flow path 360 may be capable of transferring the alveolar gas of the alveolar gas extraction line 200 to the analysis line 300 by using the flow intensity of the fluid already flowing through the analysis line 300, thereby minimizing a sudden change in the flow rate affecting the NO sensor 341.

Hereinafter, a process of measuring lung functions and screening pulmonary diseases by using the pulmonary disease screening system composed of the configurations described above will be described.

In this case, the pulmonary disease screening system may be used in a lung function measurement mode and a breath analysis mode.

First, the lung function measurement mode will be described.

### Lung Function Measurement Mode

In order to use the pulmonary disease screening system in the lung function measurement mode, only the lung function measurement line 100 may be activated, and the alveolar gas extraction line 200 and the analysis line 300 may not be operated.

Afterward, the patient may hold the mouthpiece in the mouth and exhale as hard as possible in order to measure the patient's lung function.

In this case, the first flow sensor 120 may measure the flow rate of the exhaled air that the patient exhales to the maximum.

In this way, a flow rate may be measured when the exhaled gas the patient exhales passes through the first flow sensor 120, and the flow rate measured through the first flow sensor 120 may be calculated into lung function parameters such as FVC, FEV1, FEV1/FVC, FEF 25%˜75%, PEF, and the like in the same way as the method of measuring lung functions in the existing lung function measurement devices.

Next, the breath analysis mode will be described.

### Breath Analysis Mode

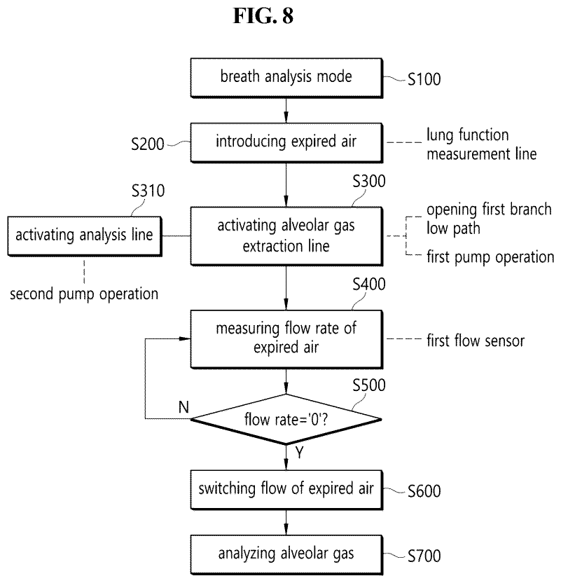

The breath analysis mode will be examined further with reference to the attached FIG. 8.

The manager may convert the pulmonary disease screening system from the lung function measurement mode to the breath analysis mode. (S100)

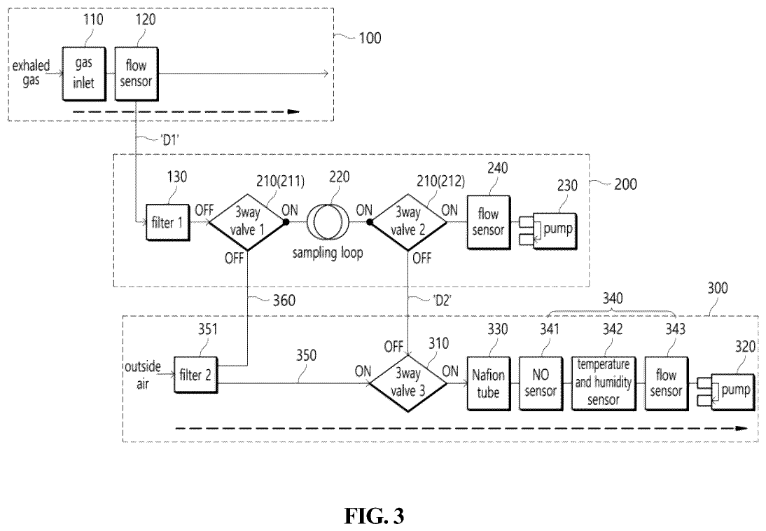

Next, the patient may hold the mouthpiece in the mouth and exhale the expired air, thereby introducing the exhaled gas into the lung function measurement line 100 as shown in FIG. 3. (S200)

As mentioned above, nitric oxide (NO) may be known as a biomarker related to pulmonary diseases such as COPD, pulmonary fibrosis, and pneumonia in exhaled gas and in order for this nitric oxide (NO) to come out in sufficient concentration through the exhalation, the patient should exhale the expired air steadily at an appropriate intensity (the flow rate), unlike in the lung function measurement mode where the patient should exhale at maximum intensity (the flow rate).

For reference, the American Thoracic Society and the European Respiratory Society recommend a flow rate of 50 ml/s as an appropriate intensity of exhalation.

In this case, when the patient exhales and directly injects the expired air into the alveolar gas extraction line 200, that is, when the filter 130 and the first valve 211 are directly connected, it may be very difficult for the patient to inject the flow rate of 50 ml/s into the alveolar gas extraction line. This may be because the orifice diameters of the first valve 211 and the second valve 212 are very small, so, only with the pressure that a person exhales and injects with, it may be difficult to inject a desired flow rate into the first valve 211.

However, the present disclosure may provide a method of extracting the expired air by using the power of the pump, so the desired flow rate of the expired air may be extracted into the alveolar gas extraction line 200 by using the first pump 230 and the first flow sensor 120 even when the patient exhales the expired air into the lung function measurement line 100 with a stable breath. An explanation thereof will be described later.

Next, the manager may activate the alveolar gas extraction line 200 and the analysis line 300. (S300)(S310)

That is, the manager may open each flow path of the alveolar gas extraction line 200 and the analysis line 300, and operate the first pump 230 and the second pump 320, thereby maintaining the gas flow in each flow path of the alveolar gas extraction line 200 and the analysis line 300.

As described above, the valve open state where the alveolar extraction gas line 200 and the analysis line 300 are activated may be as follows.

As shown in FIGS. 3 and 6, the first valve 211 of the alveolar gas extraction line 200 may be in a state where the first inlet 211a and the outlet 211c are turned ON (opened) and the second inlet 211b is turned OFF (closed). In this case, as the first inlet 211a is turned ON, the first branch flow path D1 may be opened to allow the flow path between the alveolar gas extraction line 100 and the alveolar gas extraction line 200 to be in communication with each other.

In addition, the second valve 212 of the alveolar gas extraction line 200 may be in a state where the inlet 212a and the second outlet 212c are turned ON, and the first outlet 212b is turned OFF. In this case, as the inlet 212a is turned ON, the flow path between the sampling loop 220 and the second valve 212 may be in communication with each other.

In addition, the third valve 310 of the analysis line 300 connected to the outside air inflow path 350 may be in a state where the first inlet 311a and the outlet 311c are turned ON, and the second inlet 311b is turned OFF. In this case, as the first inlet 311a is turned ON, the flow path between the outside air inflow path 350 and the third valve 310 may be in communication with each other.

Accordingly, as shown in FIG. 3, the analysis line 300 may maintain constantly a fluid

pressure in the flow path of the analysis line 300 while continuously introducing outside air through the second pump 320.

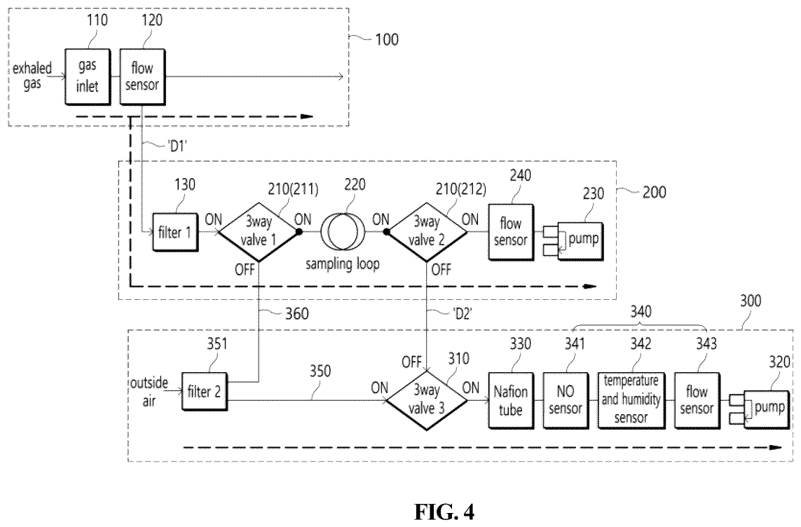

Meanwhile, as the first branch flow path D1 is opened and the alveolar gas extraction line 200 is activated, some of the expired air passing through the lung function measurement line 100 may flow into the alveolar gas extraction line 200 through the first branch flow path D1, as shown in FIG. 4.

That is, some of the expired air of the lung function measurement line 100 may sequentially pass through the filter 130, the first valve 211, the sampling loop 220, the second valve 212, and the second flow sensor 240.

Next, the manager may measure the flow rate of the expired air passing through the lung function measurement line 100 while monitoring the first flow sensor 120. (S400)

In this case, the manager may observe whether the flow rate of the expired air passing through the lung function measurement line 100 is “0” through the first flow sensor 120. This is for extracting only the alveolar gas which is the last part of the expired air, and the manager may monitor until the measured value of the first flow sensor 120 becomes “0” since the last part of the respiration contains the alveolar gas to be extracted as well as the measured value of the first flow sensor 120 rapidly decreases to “0” when the patient's respiration ends. (S500)

Next, when the flow rate of the expired air measured through the first flow sensor 120 becomes “0”, the flow of the expired air passing through the alveolar gas extraction line 200 may be switched to the analysis line 300.

At this time, as mentioned above, the fact that the flow rate of the expired air measured through the first flow sensor 120 is “0” may mean that it is the last part of the expired air when the patient's respiration ends, so, in case the flow of the expired air is switched from the alveolar gas extraction line 200 to the analysis line 300 at the moment when the flow rate of the expired air is measured as “0”, only the last part of the expired air containing the alveolar gas may remain in the sampling loop 220, such that the alveolar gas is extracted and is discharged into the analysis line 300.

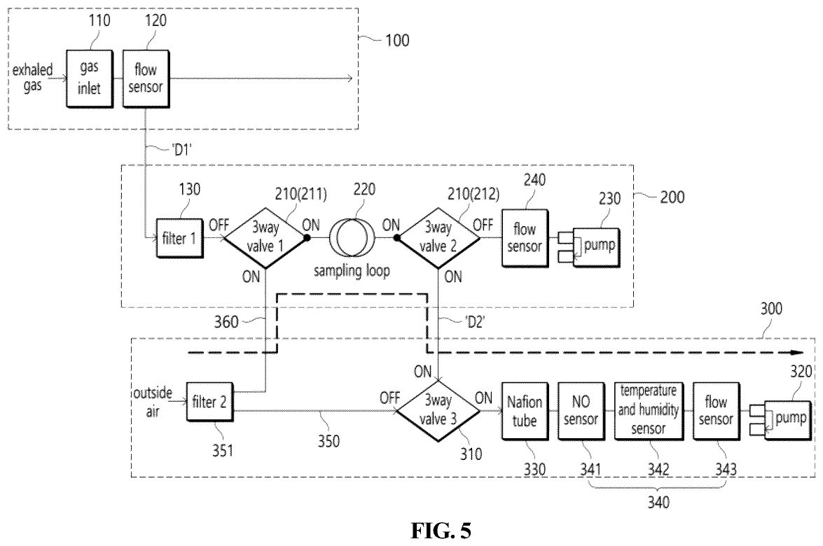

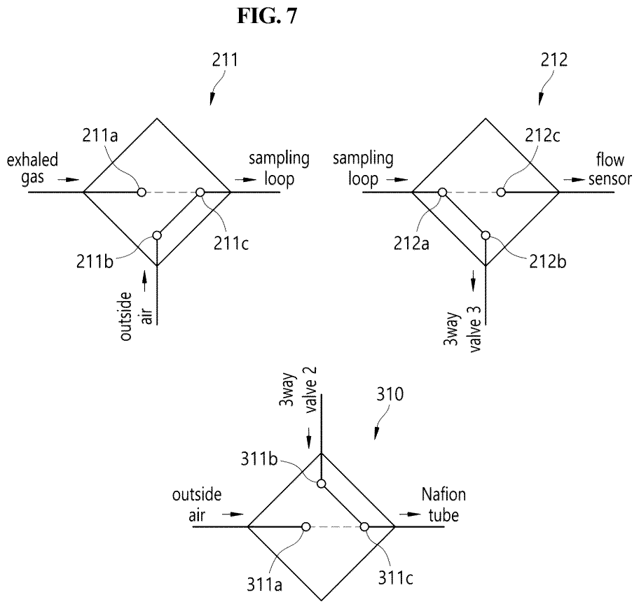

That is, as shown in FIG. 5 and FIG. 7, the first valve 211 may turn off the first inlet 211ato block communication with the lung function measurement line 100, and may tum on the second inlet 211b and the outlet 211c to convert the flow of outside air toward the sampling loop 220 through the bypass flow path 360. In addition, the second valve 212 may tum on the inlet 212a and the first outlet 212b and turn off the second outlet 212c so that the sampling loop 220 and the analysis line 300 are in communication with each other.

At this time, the third valve 310 of the analysis line 300 may tum off the first inlet 311a to block the outside air inflow path 350 and turn on the second inlet 311b and outlet 311c so that the alveolar gas extraction line 200 communicates with the analysis line 300 along with the second valve 212.

In this way, through the switching of the valve 210 and the operation of the second pump 320, the outside air passing through the flow path of the analysis line 300 may suck and transfer into the measurement unit 340 of the analysis line 300 the alveolar gas, which is the last part of the expired air, being captured in the sampling loop 220 while sequentially passing through the bypass flow path 360, the first valve 211, the sampling loop 220, the second valve 212, and the third valve 310, as shown in FIG. 5.

At this time, the intensity of the second pump 320 may be adjusted such that the alveolar gas can flow steadily to the NO sensor 341 at the flow rate recommended by the manufacturer of the NO sensor 341.

In this case, the operation of the first pump 230 may stop and as a result, may not cause any burden on the operation of the pulmonary disease screening system.

Thereafter, the moisture of the alveolar gas, that is, the last part of the expired air accommodated in the sampling loop 220 may be absorbed while passing through the Nation tube 330, and the concentration of nitric oxide, the temperature and humidity, and the flow rate may be stably measured while sequentially passing through the NO sensor 341, the temperature and humidity sensor 342, and the third flow sensor 343.

Thereafter, the manager may analyze whether any pulmonary disease such as COPD, lung fibrosis, or pneumonia is present when the concentration of nitric oxide measured through the NO sensor 341 is greater than or equal to a reference value. (S700)

As described so far, the pulmonary disease screening system and the method for screening pulmonary disease using the same may extract only the last part of the expired air the patient exhales and may measure the biomarkers in pulmonary diseases of the alveolar gas contained in the last part of the expired air, through the flow detection of the flow sensor, the expired air capture of the sampling loop, the power of the pump, and the switching control of the three-way valve.

Accordingly, the present disclosure may induce a patient's stable respiration and improve the accuracy of a pulmonary disease analysis through non-invasive pulmonary disease screening.

What is claimed is:

1. A pulmonary disease screening system, the system comprising:
a lung function measurement line forming a flow path for an expired air a patient exhales to pass through and comprising a first flow sensor for measuring a flow rate of the expired air;
a first branch flow path branched from the lung function measurement line;
an alveolar gas extraction line, which comprises a first valve connected to the first branch flow path and a first pump for generating a suction force and forms a flow path for extracting and passing a part of the expired air passing through the lung function measurement line with the suction force of the first pump;
a second branch flow path branched from the alveolar gas extraction line; and
an analysis line, which comprises a third valve connected to the second branch flow path and a second pump for generating a suction force and analyzes a biomarker in pulmonary diseases by passing an alveolar gas extracted from the alveolar gas extraction line through the second branch flow path with the suction force of the second pump,
wherein when the flow rate of the expired air measured through the first flow sensor is “0”, the first valve and the third valve are controlled to switch a flow of the expired air passing through the alveolar gas extraction line to the analysis line.

2. The system of claim 1, wherein a second valve for switching the flow of the expired air of the alveolar gas extraction line to the analysis line is further formed between the first valve and the second branch flow path.

3. The system of claim 2, wherein a sampling loop for collecting and accommodating the expired air passing through the alveolar gas extraction line is further formed between the first valve and the second valve.

4. The system of claim 3, wherein the alveolar gas extraction line further comprises a second flow sensor for measuring the flow rate of the expired air introduced into the sampling loop.

5. The system of claim 1, wherein the analysis line further forms an outside air inflow path capable of introducing outside air through the third valve.

6. The system of claim 5, wherein a bypass flow path for diverting an inflow direction of outside air to the alveolar gas extraction line is further formed between the outside air inflow path and the first valve.

7. The system of claim 1, wherein the analysis line further comprises a measurement unit for measuring a biomarker in pulmonary diseases.

8. The system of claim 7, wherein the measurement unit comprises an NO sensor for measuring nitric oxide, which is the biomarker in pulmonary diseases, a temperature and humidity sensor for measuring temperature and humidity around the NO sensor, and a third flow sensor for monitoring whether the alveolar gas flows into the NO sensor at a constant flow rate.

9. The system of claim 7, wherein a Nafion tube for reducing a humidity of the alveolar gas flowing into the measurement unit is further formed between the third valve and the measurement unit.

10. The system of claim 4, wherein when the flow rate of the expired air measured through the first flow sensor is “0”, the first valve and the second valve are controlled to switch the flow of the expired air of the alveolar gas extraction line only toward the analysis line.

11. The system of claim 6, wherein when an amount of the expired air measured through the first flow sensor is measured as “0” in a state where the third valve is controlled to close the second branch flow path and open the outside air inflow path, the first valve is controlled to open the bypass flow path, the second valve is controlled to open the second branch flow path, and the third valve is controlled to close the outside air inflow path and open the second branch flow path.

12. The system of claim 2, wherein the first valve, the second valve, and the third valve is three-way valves.

13. A pulmonary disease screening system, the system comprising:
a lung function measurement line forming a flow path for an expired air a patient exhales to pass through and comprising a first flow sensor for measuring a flow rate of the expired air;
a first branch flow path branched from the first flow sensor;
an alveolar gas extraction line comprising a first valve connected to the first branch flow path, a first pump for generating a suction force, a second valve formed between the first pump and the first valve, a sampling loop formed between the first valve and the second valve to capture an alveolar gas, a second flow sensor formed between the first pump and the second valve to measure a flow rate;
a second branch flow path branched from the second valve; and
an analysis line comprising a third valve connected to the second branch flow path, a second pump for generating a suction force, and a measurement unit formed between the third valve and the second pump to measure a biomarker in pulmonary diseases of the expired air passing through the third valve,
wherein the first valve and the second valve are controlled to extract the expired air passing through the lung function measurement line to the alveolar gas extraction line and then the first, second, and third valves are controlled to switch a flow of the expired air of the alveolar gas extraction line to the analysis line when the flow rate of the expired air measured by the first flow sensor is “0”.

14. The system of claim 13, wherein the analysis line further comprises an outside air inflow path capable of introducing outside air through the third valve, and a bypass flow path formed between the outside air inflow path and the first valve to divert an inflow direction of the outside air to the alveolar gas extraction line.

15. The system of claim 14, wherein the first valve is controlled to close the first branch flow path and open the bypass flow path, the second valve is controlled to allow the expired air to be discharged only toward the second branch flow path, and the third valve is controlled to close the outside air inflow path and open the second branch flow path,
when the flow rate of the expired air is measured as “0” through the first flow sensor in a state where the first valve and the second valve are controlled to open only the first branch flow path and the flow path of the alveolar gas extraction line and the third valve is controlled to open only the outside air inflow path and the analysis line.

16. A pulmonary disease screening method for extracting an expired air a patient exhales and analyzing the expired air, wherein when a flow rate of the expired air becomes “0”, the method performs pulmonary disease screening by extracting an alveolar gas, which is the last part of the expired air, and by analyzing a biomarker in pulmonary diseases.

17. The method of claim 16, wherein an alveolar gas extraction from the expired air is performed through a flow detection of a flow sensor, an expired air capture of a sampling loop, a power of a pump, and a switching control of a valve.

18. The method of claim 17, the method comprising:
(a) injecting the expired air the patient exhales into a lung function measurement line and continuously introducing outside air into an analysis line;
(b) measuring the flow rate of the expired air the patient exhales into the lung function measurement line by a manager;
(c) extracting the expired air passing through the lung function measurement line into an alveolar gas extraction line;
(d) discharging an alveolar gas extracted from the alveolar gas extraction line to the analysis line by switching the flow of the expired air of the alveolar gas extraction line to the analysis line and by diverting a flow of outside air to the alveolar gas extraction line by the analysis line when the flow rate of the expired air becomes “0” in the step (b); and
(e) analyzing the biomarker in pulmonary diseases in the alveolar gas introduced from the alveolar gas extraction line by the analysis line.
